(19) United States
(12) Patent Application Publication
(10) Pub. No.: US 2026/0027064 A1
(43) Pub. Date: Jan. 29, 2026

(54) METHODS OF MAKING AND USING NANOPARTICLES FOR TREATMENT OF BACTERIAL BIOFILM

(71) Applicant: Brown University, Providence, RI (US)

(72) Inventors: Anita Shukla, East Greenwich, RI (US); Zhaowei Jiang, Providence, RI (US)

(21) Appl. No.: 19/280,577

(22) Filed: Jul. 25, 2025

Related U.S. Application Data
(60) Provisional application No. 63/675,469, filed on Jul. 25, 2024.

(51) Int. Cl. A61K 9/51; A61K 9/00; A61K 38/46; A61P 31/04

(52) U.S. Cl. CPC A61K 9/5146; A61K 9/0014; A61K 9/5169; A61K 9/5192; A61K 38/465; A61P 31/04

(57) ABSTRACT

A method of making and using nanoparticles that inhibit formation of bacterial biofilm are provided.

### RELATED APPLICATION DATA

This application claims priority to U.S. Ser. No. 63/675,469 filed Jul. 25, 2024 hereby incorporated by reference in its entirety for all purposes.

### STATEMENT OF GOVERNMENT INTERESTS

This invention was made with government support under N00014-22-1-2336 awarded by the Office of Naval Research. The government has certain rights in the invention.

### FIELD

The present invention relates in general to the design and use of polymer coated nanoparticles to adversely affect bacterial biofilm formation.

### BACKGROUND

More than 2.8 million antimicrobial-resistant infections occur each year in the United States and over 80% of microbial infections are due to biofilms, many of which are resistant to standard anti-microbial treatments and can require surgery. See Centers for Disease Control and Prevention (U.S.) Antibiotic resistance threats in the United States, 2019; C\acute{ }amara, M.; Green, W.; MacPhee, C. E.; Rakowska, P. D.; Raval, R.; Richard-son, M. C.; Slater-Jefferies, J.; Steventon, K.; Webb, J. S. Economic significance of biofilms: a multidisciplinary and cross-sectoral challenge. npj Biofilms and Microbiomes 2022, 8, 1-8.

Staphylococcus epidermidis, a major healthcare-associated bacterium, commonly causes infections related to implanted medical devices like catheters and prostheses. In fact, S. epidermidis represents the most common source of infections on indwelling medical devices. See Otto, M. Staphylococcus epidermidis—the ‘accidental’ pathogen. Nature Reviews Microbiology 2009, 7, 555-567. Consequently, physicians must remove and replace the infected device, which increases both the cost and morbidity for patients. See Fey, P. D.; Olson, M. E. Current concepts in biofilm formation of Staphylococcus epidermidis. Future Microbiology 2010, 5, 917-933. Furthermore, the formation of biofilms by S. epidermidis on these devices complicates treatment, as biofilms exhibit both resistance and tolerance to antibiotics, making them difficult to eradicate.

Staphylococcus epidermidis exists as a commensal organism on the skin. This opportunistic pathogen becomes harmful when introduced to the body via medical devices. See Allen-Taylor, D.; Boro, G.; Cabato, P. M.; Mai, C.; Nguyen, K.; Rijal, G. Staphylococcus epidermidis biofilm in inflammatory breast cancer and its treatment strategies. Biofilm 2024, 8, 100220. On the medical device surfaces, Staphylococcus epidermidis biofilms form and colonize, protecting against the host immune response and antibiotic treatments. Staphylococcus epidermidis can bind specifically to host proteins present in the skin because of its commensal nature. Within a surgical wound, the bacterium leverages these adhesion mechanisms to attach to deeper tissues and implanted devices, particularly to the layer of host proteins that accumulate on the device's surface. See Sabat\acute{ }e Bresc\acute{ }o, M.; Harris, L. G.; Thompson, K.; Stanic, B.; Morgenstern, M.; O'Mahony, L.; Richards, R. G.; Moriarty, T. F. Pathogenic Mechanisms and Host Interactions in Staphylococcus epidermidis Device-Related Infection. Frontiers in Microbiology 2017, 8. This ability by Staphylococcus epidermidis is a virulence factor that initiates biofilm formation.

Bacterial biofilms are complex microbial communities encased in extracellular polymeric substances (EPS), involve a multistep formation process, and are one of the primary reasons for the persistence of infections. See Vestby, L. K.; Gronseth, T.; Simm, R.; Nesse, L. L. Bacterial Biofilm and its Role in the Pathogenesis of Disease. Antibiotics 2020, 9, 59. The EPS consists of proteins, polysaccharides, extracellular DNA, and lipids. Because of this matrix environment, the bacteria change their metabolic rates in order to become more resistant to antimicrobial agents. See Vestby, L. K.; Gronseth, T.; Simm, R.; Nesse, L. L. Bacterial Biofilm and its Role in the Pathogenesis of Disease. Antibiotics 2020, 9, 59.

Biofilm-related infections, such as Staphylococcus epidermidis and Pseudomonas aeruginosa, pose a challenge to eradicate. Therefore, methods and materials are needed for the treatment of bacterial infections which can overcome the resistance provided by the bacterial biofilm.

### SUMMARY

Aspects of the present disclosure are directed to nanoparticles having a nanoparticle core and a polymer conjugated thereto by an acid labile bond and methods of using such nanoparticles to treat or prevent or inhibit bacterial biofilm formation. According to one aspect, inhibiting or preventing bacterial biofilm formation inhibits or prevents bacterial proliferation. According to one aspect, nanoparticles described herein inhibit or prevent adhesion of bacteria to a substrate surface, and therefore inhibit or prevent the abiliy of bacteria to generate a bacterial biofilm. According to one aspect, nanoparticles describe herein inhibit or prevent adhesion of bacterial biofilm to a substrate surface, and therefore inhibit or prevent bacterial proliferation. Without wishing to be bound by scientific theory, the nanoparticles described herein are believe form a barrier or layer or coating or a collection of nanoparticles in general between bacteria and a substrate surface. When such a barrier or layer or coating or collection of nanoparticles are present between bacteria and a substrate surface, the bacteria is inhibited or prevented from adhering to the substrate surface. According to one aspect, the low pH environment of bacteria and gelatinase created by bacteria cause the nanoparticles of the present disclosure to degrade thereby inhibiting or preventing adhesion of the bacteria to the substrate surface.

According to one aspect, the nanoparticles prevent or inhibit adhesion of bacteria or bacterial biofilm to a substrate thereby preventing or inhibiting proliferation of bacteria or bacterial biofilm on the substrate. In this manner, a barrier of nanoparticles, which may be a layer or coating of nanoparticles or a collection of nanoparticles is provided on a substrate, such as an implant or tissue. Bacteria attempting to form a biofilm on the layer or coating of nanoparticles will generate an acidic pH and/or a gelatinase. As a result of the acidic pH, the polymer will be released from the nanoparticle core via acid hydrolysis of the acid labile bond, thereby destabilizing, disrupting or breaking the barrier or layer or coating or collection of nanoparticles onto which the bacteria is attempting to form a biofilm. The destabilizing, disrupting or breaking of the barrier or layer or collection of nanoparticles on the substrate prevents or inhibits adhesion of the bacteria or bacterial biofilm to the substrate, thereby preventing or inhibiting biofilm formation and proliferation of bacteria. For example, bacteria or biofilm contacting the barrier or layer or coating or collection of nanoparticles including the acid-releasable polymer will be dislodged from the substrate as a result of the polymer being released from the substrate surface due to breakage of the acid labile bond.

According to one aspect, nanoparticles described herein have a gelatin nanoparticle core to which the polymer is attached, bound, linked or conjugated via an acid labile bond. As a result of the gelatinase produced by the bacteria, the gelatin core will be degraded thereby destabilizing, disrupting or breaking the barrier or layer or coating or collection of nanoparticles on the substrate onto which the bacteria is attempting to form a biofilm. The destabilizing, disrupting or breaking of the barrier or layer or coating or collection of nanoparticles on the substrate prevents or inhibits adhesion of the bacteria or bacterial biofilm to the substrate, thereby preventing or inhibiting biofilm formation and proliferation of bacteria. For example, bacteria or biofilm contacting the barrier or layer or collection of nanoparticles including the gelatin nanoparticle core will be dislodged from the substrate as a result of the gelatin nanoparticle core being degraded and portions of which being released from the substrate surface.

According to one exemplary aspect, the layer or coating of nanoparticles becomes unstable due to release of the polymer and/or the degradation of the gelatin nanoparticle core. Without wishing to be bound by scientific theory, the instability of the layer or coating of nanoparticles inhibits or prevents adhesion of the bacteria or bacterial biofilm to the substrate.

Nanoparticles as described herein may be referred to herein shorthand as “P-GNP” meaning that a polymer is attached, bound, linked or conjugated to a nanoparticle core by an acid labile bond. The polymer may be in the form of a coating on the nanoparticle core. The “GNP” refers specifically to a gelatin nanoparticle core, however it is to be understood that the nanoparticle core may be made of substances other than gelatin or may include substances other than gelatin. It is to be understood that the nanoparticle core may be degradable or non-degradable.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-1B. Schematic representation of P-GNP. FIG. 1A. Schematic showing detachment of polymer from the nanoparticle core in response to an acidic environment and degradation of gelatin nanoparticle cores forming portions of the cores leading to disruption or destabilization of the collection of P-GNP at the substrate surface leading to inhibition or prevention of bacterial adhesion and biofilm adhesion. FIG. 1B. Synthesis steps to prepare P-GNP.

FIGS. 2A-2E. Nanoparticle characterizations. FIG. 2A. Hydrodynamic diameter of NPs in miliQ water; FIG. 2B. TEM imaging of GNP and FIG. 2C. P-GNPs, respectively; Scale bar=200 nm; FIG. 2D. FTIR spectrum of the copolymer, GNP, and P-GNP; FIG. 2E. Zeta-potential of the NPs in milliQ water. Results are shown as mean±standard deviation. Statistical significance is indicated by matching using two-way ANOVA with multiple comparisons with Dunnett's test (*** indicates p<0.001 and **** indicates p<0.0001), with n=3.

FIGS. 3A-3C. Antibacterial and antibiofilm efficacy in a biofilm inhibitory assay of a barrier or layer or collection of P-GNPs between a substrate and bacteria compared to control groups where bacteria and P-GNPs are added together into a well substrate. FIG. 3A. Normalized biofilm viability (%) of S. epidermidis treated with P-GNPs and copolymer controls. FIG. 3B. Normalized bacterial growth (%) of S. epidermidis treated with P-GNPs and copolymer controls at varying concentrations. FIG. 3C. Scanning electron microscopy (SEM) images of S. epidermidis biofilms after treatment with 1×PBS (negative control), copolymer, GNP, and P-GNP. Results are shown as mean±standard deviation. Statistical significance is indicated by matching using two-way ANOVA with multiple comparisons with Dunnett's test (*** indicates p<0.001 and *** indicates p<0.0001), with n=3. Images are representative of at least three independent experiments per NP formulation. Scale bars are 5 μm. P-GNP is able to inhibit or prevent bacterial adhesion and biofilm formation as demonstrated by a decrease of biofilm viability post washing, i.e., less biofilm remains on the substrate surface.

FIGS. 4A-4B. Effect of nanoparticle treatments on biofilm structure and bacterial viability where bacteria and P-GNP are added together and incubated overnight, followed by washing to remove free floating bacteria and staining and imaging. FIG. 4A. Representative confocal laser scanning microscopy (CLSM) images of bacterial biofilms after treatment with different nanoparticle formulations: No Treatment, GNP, PEG-PEI, and P-GNP. Biofilms were stained with SYTO9 (green), indicating live bacteria. Scale bars: 100 μm (white for x- and y-projection) and 20 μm (for z-projection). FIG. 4B. Three-dimensional reconstructions of biofilms stained with LIVE/DEAD BacLight bacterial viability kit. Red fluorescence indicates dead or membrane-compromised bacteria.

FIGS. 5A-5B. In vitro cytotoxicity and hemolysis of NP formulations. FIG. 5A. Cytotoxicity of NP formulations with NIH 3T3; FIG. 5B. Human red blood cell hemolysis upon incubation with NP formulations. Results are shown as mean±standard deviation with n=3.

FIGS. 6A-6C. Evaluation of P-GNP antibacterial efficacy in an ex vivo porcine skin burn wound model infected with S. epidermidis. FIG. 6A. Schematic illustration of study design. Image created with Biorender.com FIG. 6B. Normalized viability (%) of S. epidermidis in burn wounds treated with different concentrations of P-GNP. FIG. 6C. Cell forming unit per mL of media upon prophylactic PGNP treatment. Statistical significance is indicated by matching using two-way ANOVA with multiple comparisons with Dunnett's test (**** indicates p<0.0001), with n=3.

FIGS. 7A-7C. Evaluation of P-GNP antibacterial efficacy in antibacterial adhesion in an implant model infected with S. epidermidis. FIG. 7A. Schematic illustration of study design. Image created with Biorender.com FIG. 7B. Normalized viability (%) of S. epidermidis on implant upon prophylactic treatment with P-GNP. FIG. 7C. Cell forming unit per mL of media upon prophylactic P-GNP treatment. Statistical significance is indicated by matching using two-way ANOVA with multiple comparisons with Dunnett's test (**** indicates p<0.0001), with n=3.

FIG. 8. Minimal biofilm inhibitory assay using P-GNP against P. aeruginosa PA01. Statistical significance is indicated by matching using two-way ANOVA with multiple comparisons with Dunnett's test (* indicates p<0.01; and indicates p<0.0001), with n=3.

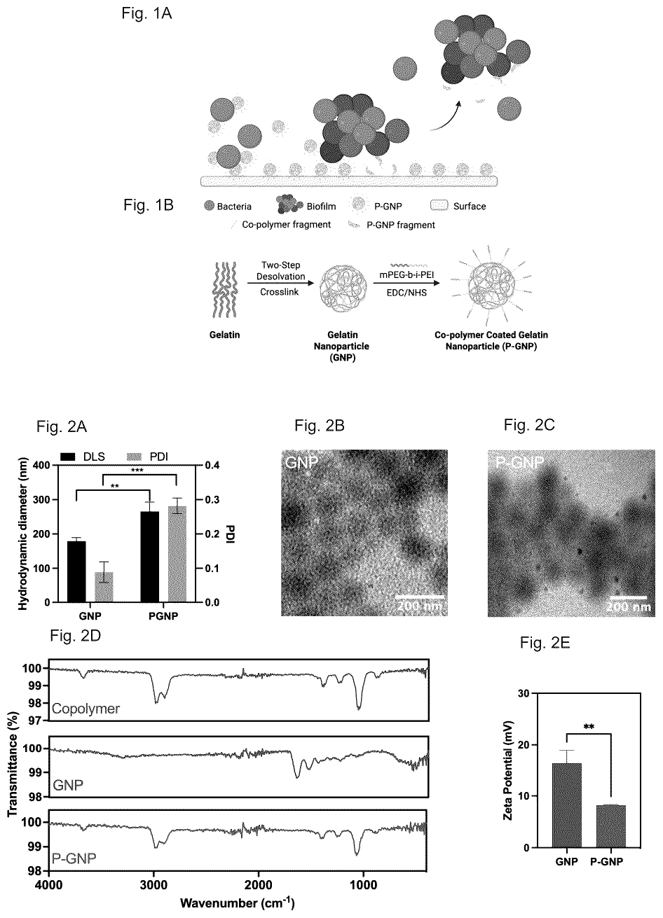

### DETAILED DESCRIPTION

The present disclosure provides methods of designing and using nanoparticles to inhibit or prevent formation of a bacterial biofilm, such as by preventing adhesion of bacteria or bacterial biofilm to a substrate surface. According to one aspect, the nanoparticles described herein are effective to inhibit or otherwise prevent bacterial adhesion and biofilm formation. According to one aspect, the nanoparticles described herein are effective to inhibit or prevent growth of bacterial biofilms. According to one aspect, the nanoparticles described herein are effective to reduce the presence of bacterial biofilms that inhibits or prevents proliferation of bacetriaor or otherwise destroys or eradicates bacterial biofilms. According to one exemplary aspect, the nanoparticles described herein lack an antimicrobial agent, such as within the nanoparticle core or a polymer layer, that is toxic to the bacteria. According to one exemplary aspect, the nanoparticles described herein exclude an antimicrobial agent, such as within the nanoparticle core or a polymer layer, that is toxic to the bacteria. According to one exemplary aspect, nanoparticles as described herein are provided with the proviso that they do not include an antimicrobial agent, such as within the nanoparticle core or a polymer layer, that is toxic to the bacteria.

According to one aspect, nanoparticles described herein include a polymer attached, bound, linked or conjugated to a nanoparticle core by an acid labile bond. The polymer may be attached in the form of a layer or coating, i. a plurality of polymer molecules, on or otherwise surrounding the nanoparticle core. Acid labile bonds useful in the present disclosure are readily identifiable to one of skill based on the present disclosure. According to one aspect, the acid labile bond is susceptible to being broken within the environment of an acidic pH such as that produced by bacteria. One such mechanism is acid hydrolysis of the acid labile bond. Other mechanisms of breaking an acid labile bond will become apparent to those of skill based on the present disclosure. Once the acid labile bond is broken, the polymer becomes releasable from the nanoparticle core and may migrate away from the nanoparticle core. According to one aspect, the polymer may be a copolymer, terpolymer etc. For ease of understanding of the present disclosure, the term polymer is a genus that includes a copolymer, terpolymer etc. According to one aspect, the polymer may be referred to herein as a “pH-responsive polymer” insofar as the polymer is released upon breakage of the bond between the polymer and the nanoparticle core.

According to one aspect, the nanoparticle core includes gelatin. The gelatin is susceptible to being degraded by a gelatinase, such as a gelatinase produced by bacteria. As a result, the nanoparticle core including gelatin may be degraded into smaller portions. Once degraded into smaller portions, the smaller portions may migrate away from each other.

According to one exemplary aspect, the nanoparticles described herein may be at a substrate surface as a layer or barrier or collection using methods known to those of skill in the art and that may become apparent based on the present disclosure. The substrate is one in which it is desired to inhibit or prevent bacterial proliferation. The substrate may include an implant or implantable material as are known in the art. The substrate may include tissue. Bacteria comes in contact with the barrier or layer or collection of nanoparticles and attempts to form a biofilm. For ease of understanding, reference will be to a layer, but it is to be understood that a barrier or collection of nanoparticles can also be used. The layer is provided on the substrate in order to inhibit or prevent adhesion of bacteria or bacterial biofilm thereto. According to one aspect, when adhesion of bacteria or bacterial biofilm is inhibited or prevented, bacterial proliferation is inhibited or prevented. According to one aspect, methods are described herein of using a layer of the nanoparticles as described herein on a substrate to inhibit adhesion of the bacteria or bacterial biofilm to the substrate. In this manner, the bacteria produce an acidic pH when attempting to proliferate and produce a bacterial biofilm on the surface of the substrate. According to one aspect, the acid labile bonds between the polymer and nanoparticle core break in response to the acidic pH and the polymer is susceptible to migration away from the substrate surface insofar as the polymer is released from the substrate surface. Once the acid labile bond is broken, the polymer becomes releasable from the nanoparticle core and may migrate away from the nanoparticle core. According to one aspect, any bacteria or bacterial biofilm contacting or adjacent to or near the released copolymer will also be susceptible to migration away from the substrate surface. In this exemplary manner, the layer becomes destabilized or disrupted and inhibits or prevents adhesion of the bacteria or bacterial biofilm to the substrate surface insofar as a portion of the layer is removed and bacteria or bacterial biofilm contacting or adjacent to or near the layer may similarly be removed away from the substrate surface.

According to one aspect, the nanoparticle forming the layer includes a nanoparticle core that includes gelatin. The gelatin is susceptible to being degraded by a gelatinase produced by bacteria attempting to proliferate and create a bacterial biofilm. According to one exemplary aspect, the nanoparticle core including gelatin may be degraded into smaller portions and the smaller portions migrate away from the substrate surface. According to one aspect, any bacteria or bacterial biofilm contacting or adjacent to or near the degraded nanoparticle core will also be susceptible to migration away from the substrate surface. In this exemplary manner, the layer becomes destabilized or disrupted and inhibits or prevents adhesion of the bacteria or bacterial biofilm to the substrate surface insofar as a portion of the layer is removed and bacteria or bacterial biofilm contacting or adjacent to or near the layer may similarly be removed from the substrate surface.

While the nanoparticles are described herein as including two principle aspects of a polymer attached to a nanoparticle core via an acid labile bond, wherein the nanoparticle core may include a gelatin, it is to be understood that the nanoparticle may include other components within the polymer layer or nanoparticle core as described herein, and consistent with the function of the nanoparticle to be degraded by either acid hydrolysis of the acid labile bonds connecting the polymer to the nanoparticle core and/or to be degraded by a gelatinase degrading gelatin which may be present in the nanoparticle core.

According to aspects described herein, nanoparticles are designed and used to treat bacterial biofilms in a manner to prevent and/or inhibit and/or eradicate bacterial biofilms, such as those present on a substrate surface. The nanoparticles are designed to be responsive to one or more enzymes, such as gelatinase, or one or more conditions of the biofilms, such as pH. For example, the bond between the polymer and the nanoparticle core is selected to be responsive to the acidic pH of the environment of bacteria or biofilm, so as to disengage the polymer from the nanoparticle core. For example, the nanoparticle may include gelatin to be responsive to the gelatinase enzyme present in the environment of the bacteria or biofilm, so as to be degraded by the gelatinase, thereby degrading the nanoparticle core. According to one aspect, the polymer coated nanoparticles as described herein inhibit or prevent bacterial growth without the need for an antibacterial agent toxic to the bacteria, by inhibiting bacterial adhesion and biofilm adhesion and formation, and thus proliferation of bacteria. According to one aspect, the bacteria die, and therefore cease to produce bacterial biofilm. As a result, the bacterial biofilm is destroyed or otherwise eradicated. According to one aspect, the polymer coated nanoparticles lack an antibacterial agent that is itself toxic to the bacteria. Though, it is to be understood that such an antibacterial agent that is itself toxic to the bacteria, may be present in the polymer coated nanoparticles.

### I. Exemplary Bacteria

Bacteria according to the present disclosure includes any bacteria which creates a biofilm and which generates an acidic environment (such as a low pH). Exemplary bacteria may also produce a gelatinase. It is to be understood that the basic concepts of the present disclosure described herein are not limited by bacteria type. Bacteria according to the present disclosure may include one or members of the species Vibrio, Clostridium, Escherichia, Rhodococcus, Pseudomonas, Bacillus, Lactobacillus Saccharomyces, Acinetobacter, Staphylococcus, Enterobacter, Klebsiella and Enterococcus.

Exemplary genus and species of bacteria cells for use in the methods described herein include Acetobacter aurantius, Acinetobacter bitumen, Actinomyces israelii, Agrobacterium radiobacter, Agrobacterium tumefaciens, Anaplasma Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella,Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

According to one aspect, exemplary bacteria include Staphylococcus epidermis. According to one aspect, exemplary bacteria include Pseudomonas aeruginosa.

### II. Exemplary Bacterial Conditions or Environments

The present disclosure recognizes that the microenvironment of bacterial infection sites is unlike that in normal tissue in terms of the concentration or composition of various substances. See D. Hu, Y. Deng, F. Jia, Q. Jin and J. Ji, ACS Nano, 2020, 14, 347-35; C. Wang, W. Zhao, B. Cao, Z. Wang, Q. Zhou, S. Lu, L. Lu, M. Zhan and X. Hu, Chemistry of Materials, 2020, 32, 7725-7738; Y. Liu, A. Lin, J. Liu, X. Chen, X. Zhu, Y. Gong, G. Yuan, L. Chen and J. Liu, ACS Applied Materials & Interfaces, 2019, 11, 26590-26606; D. Alkekhia, H. Safford, S. Shukla, R. Hopson and A. Shukla, Chem. Commun., 2020, 56, 11098-11101; D. Pornpattananangkul, L. Zhang, S. Olson, S. Aryal, M. Obonyo, K. Vecchio, C.-M. Huang and L. Zhang, Journal of the American Chemical Society, 2011, 133, 4132-4139.

For example, lower pH is often found at bacterial infection sites. See S. Fulaz, D. Hiebner, C. H. N. Barros, H. Devlin, S. Vitale, L. Quinn and E. Casey, ACS Applied Materials & Interfaces, 2019, 11, 32679-32688. The localized acidic biofilm microenvironment results from the production of acidic by-products during bacterial metabolism. See A. M. Scharnow, A. E. Solinski and W. M. Wuest, Med. Chem. Commun., 2019, 10, 1057-1067. Overexpression of proteases also occurs at the infection sites. See G.-B. Qi, D. Zhang, F.-H. Liu, Z.-Y. Qiao and H. Wang, Advanced Materials, 2017, 29, 1703461; L.-L. Li, H.-L. Ma, G.-B. Qi, D. Zhang, F. Yu, Z. Hu and H. Wang, Advanced Materials, 2016, 28, 254-262; X. Wang, J. Wu, P. Li, L. Wang, J. Zhou, G. Zhang, X. Li, B. Hu and X. Xing, ACS Applied Materials & Interfaces, 2018, 10, 34905-34915. For example, bacterial gelatinases contribute to biofilm formation and virulence through degradation of a broad range of host substrates. See L. R. Thurlow, V. C. Thomas, S. Narayanan, S. Olson, S. D. Fleming and L. E. Hancock, Infection and Immunity, 2010, 78, 4936-4943. Another class of common enzyme found at the infection sites are hyaluronidases which are virulence factors that are involved in the invasion, and penetration of tissues in bacterial infection. See W. L. Hynes and S. L. Walton, FEMS Microbiology Letters, 2000, 183, 201-207.

According to the present disclosure, nanoparticles are designed based on these biofilm environment features using materials responsive to these biofilm environment features, such as a nanoparticle core including gelatin and pH-degradable bonds or linkages or conjugations between a polymer and a nanoparticle core.

As described herein, the exemplary bacteria S. epidermis or P. aeruginosa create a biofilm. The biofilm is characterized by an acidic pH and optionally the presence of proteases, such as gelatinases. An exemplary biofilm may be characterized by both an acidic pH and the presence of a gelatinase. As described herein, nanoparticles are described that respond to the bacterial enzymes and pH. For example, a material (not necessarily a polymer) is connected to a nanoparticle core via an acid labile bond which can be broken or otherwise which can be degraded in an acidic pH thereby releasing the material from the nanoparticle core. A material which can be degraded by a gelatinase can be used, such as gelatin. One of skill can design the nanoparticle core and materials attached thereto to be responsive to one or more conditions of the bacterial biofilm.

### III. Exemplary Polymers for Attaching to a Nanoparticle Core

According to certain aspects, a polymer is attached, bound, linked or conjugated to a nanoparticle core. The term “polymer” is intended as a genus to include copolymers, terpolymers etc. An exemplary polymer is a poly(ethylene glycol) (PEG) derivative. An exemplary polymer is a copolymer of PEG and polyethyleneimine (PEI). An exemplary polymer is a pegylated branched polyethylenimine copolymer. Certain copolymers have been known to exhibit antimicrobial activity. See Lam et al., PEGylation of Polyethylenimine Lowers Acute Toxicity while Retaining Anti-Biofilm and -Lactam Potentiation Properties against Antibiotic-Resistant Pathogens. ACS Omega 2020, 5, 26262-26270, hereby incorporated by reference in its entirety. However, such pegylated branched polyethylenimine copolymers are utilized in high concentration. According to one aspect of the present disclosure, polymers described herein are present in an amount that is nontoxic to the bacteria present in a bacterial biofilm.

Exemplary polymers include methoxy PEG-benzoic-imine-branched polyethyleneimine, poly(l-lysine)-based copolymers, poly(quaternary ammonium) copolymers, polyguanidine copolymers, poly(vinylpyridine) derivatives, polyethylene glycol-poly(lactic acid), poly(ε-caprolactone), chitosan-g-PEG, Poly (β-amino ester) copolymers, polypeptide-based copolymers, chitosan-based copolymers, gelatin-PEG copolymers, alginate-based copolymers, cellulose-based copolymers, ROS-generating copolymers, poly(carboxybetaine)-based copolymers, poly(sulfobetaine methacrylate) (PSBMA) copolymers and charge-convertible copolymers.

It is to be understood that the above list is not exhaustive. One of skill will readily be able to identify exemplary polymers that can be connected to a nanoparticle core via an acid labile bond.

### IV. Exemplary Acid Labile Bonds

According to one aspect, an acid labile bond connects the polymers described herein to the nanoparticles described herein. An exemplary acid labile bond is an amide bond that is subject to acid hydrolysis as is known in the art. Additional acid labile bonds include Hydrazone Bonds (R₁—C═NNH—R₂), Schiff Base (Imine) Bonds (R₁—C═NR₂), Acetal/Ketal Bonds (R₂C(OR′)₂ or R₂C (OR′)(OR″)), Cis-Aconityl Linkage (R₁—CO—CH═CH—CO-R₂), Ortho Ester Bonds (R—C(OR′)₃), Ester Bonds (R—COOR′), β-Thiopropionate Bonds (R—CO—CH₂—S—R′), Boronate Esters (R—B(OR′)₂), Oxime Bonds (R₁—C═N—OH—R₂), Maleic Acid Amide Bonds (maleamic acid), Silyl Ether Bonds (R—O—SiR′₃), β-Carboxylic Acid Amides (R₁—CH₂—CH(CO₂H)—NH—R₂), β-Thioketal Bonds (R—CH(SR')—CH(SR″)—R), Boronic Acid Bonds (R—B(OH)₂), Boronic Ester (R—B(OR′)₂), Disulfide Bonds (R₁—S—S—R₂), Proline-Thioketal Linkers and the like. It is to be understood that this list is not exhaustive and that other acid labile bonds will become apparent to one of skill based on the present disclosure.

### V. Exemplary Nanoparticle Cores

The present disclosure provides the use of a nanoparticle core in the nanoparticles described herein. Nanoparticle cores may include materials known to those of skill in the art of nanoparticle manufacture. Such materials may include polymers.

Such materials may include gelatin. According to the present disclosure, gelatin possesses excellent bio compatibility and biodegradability. Advantageously, gelatin will not cause adverse reactions with living tissue because it will naturally break down within the body. Gelatin is also versatile, meaning it can be utilized in a wide range of applications, such as hydrogels or nanoparticles. In comparison to other naturally derived polymers, gelatin exhibits better flexibility and mechanical strength, especially when used with cross-linking agents. Gelatin nanoparticles (GNPs) can be easily manipulated in terms of their size, surface charge, and drug release profile. GNPs can also target specific locations in the body, maximizing the amount of pH responsive copolymer reaching the area of infection and minimizing exposure and side effects to healthy tissue.

Due to the production of gelatinases by many common biofilm pathogens and at infection sites, the gelatin nanoparticles degrade at the site of infection to release smaller portions of the nanoparticle thereby releasing the polymer attached thereto.

According to one aspect, the pH-responsive copolymer is conjugated to the gelatin nanoparticles, and may or may not be in the form of a coating. According to one aspect, a pH-degradable agent, such as a poly(ethylene glycol) (PEG) derivative, is conjugated to the gelatin nanoparticle to form a coating. The pH-responsive coating detaches from the gelatin nanoparticle in the presence of low pH at the bacteria or biofilm site. The present disclosure provides a dual-stimuli responsive (i.e., pH and enzyme-responsive) nanoparticle to treat bacterial biofilms by locally delivering an antimicrobial or antibacterial effective amount of a pH-responsive copolymer coated gelatin nanoparticle.

Gelatin nanoparticles used herein may be those described in drug delivery systems such as P. S. O. Victoria and Speiser, U.K Patent GB1516348, 1978 and R. C. Oppenheim, J. J. Marty and P. Speiser, U.S. Pat. No. 4,107,288, each of which is hereby incorporated herein by reference in its entirety. Various surface modifications which may be useful to make or modify the nanoparticles described herein are described in A. Lin, Y. Liu, X. Zhu, X. Chen, J. Liu, Y. Zhou, X. Qin and J. Liu, ACS Nano, 2019, 13, 13965-13984; L.-L. Li, J.-H. Xu, G.-B. Qi, X. Zhao, F. Yu and H. Wang, ACS Nano, 2014, 8, 4975-4983; J. Su, R. Zhang, Y. Lian, Z. Kamal, Z. Cheng, Y. Qiu and M. Qiu, Pharmaceutics, 2019, 11, 93; S. Kirar, N. S. Thakur, J. K. Laha and U. C. Banerjee, ACS Applied Bio Materials, 2019, 2, 4202-4212; S. Balthasar, K. Michaelis, N. Dinauer, H. v. Briesen, J. Kreuter and K. Langer, Biomaterials, 2005, 26, 2723-2732; T. G. Shutava, S. S. Balkundi, P. Vangala, J. J. Steffan, R. L. Bigelow, J. A. Cardelli, D. P. O'Neal and Y. M. Lvov, ACS Nano, 2009, 3, 1877-1885; X.-h. Tian, F. Wei, T.-x. Wang, D. Wang, J. Wang, X.-n. Lin, P. Wang and L. Ren, Materials Letters, 2012, 68, 94-96 each of which is hereby incorporated herein by reference in its entirety.

### VI. Exemplary Nucleases

According to one aspect, the nanoparticle optionally includes a nuclease, such as DNase I, which may be attached to the surface of the nanoparticle core (which may be in the form of a layer or coating) which degrades extracellular DNA of the bacterial biofilm. According to one aspect, the polymer is provided as a coating over the nuclease. The polymer coating is attached to the nanoparticle core via acid labile bonds. At the site of the bacterial biofilm, the acid labile bonds break and release the polymer exposing the nuclease, if present, which then degrades extracellular DNA present in the biofilm.

### VII. Exemplary Nanoparticles Having Cores and Layers

Exemplary nanoparticles as described herein have a diameter of between 1 and less than 1000 nanometers, between 1 and 500 nm, between 1 and 250 nm, between 1 and 200 nm, between 1 and 150 nm, between 1 and 100 nm although nanoparticles can also have diameters suitable for the particular application. Nanoparticles can have diameters above 100 nanometers and below 1 nanometer. Nanoparticles are usually present as a distribution of diameters.

The nanoparticle may include a layer or coating on or otherwise surrounding the nanoparticle core, such as a polymer layer or coating. It is to be understood that the term nanoparticle includes a particle having a core and may have one or more layers. According to one aspect, the nanoparticle includes a core and one or more, two or more or a plurality of layers.

### VIII. Exemplary Substrates

According to one aspect of the present disclosure, a layer of the nanoparticles described herein may be provided on a substrate where inhibition or prevention of bacterial proliferation is desired. A layer of nanoparticles can be provided on a substrate using any method known to those of skill in the art including spraying or otherwise placing a liquid formulation including the nanoparticles on the surface of the substrate and allowing to dry so as to form the layer.

Exemplary methods of providing or forming a layer of nanoparticles include dip coating, spin coating, spray coating, layer-by-layer (LbL) assembly, electrostatic adsorption, drop casting, inkjet printing, electrophoretic deposition, plasma-assisted deposition, chemical vapor deposition (CVD), can be adapted based on intended application (e.g., medical implants, wound dressings, catheters. Post-deposition crosslinking, annealing, or surface curing steps may also be employed to enhance coating stability or functionality and the like. It is to be understood that this list is not exhaustive and that other methods of creating a layer will become apparent to one of skill based on the present disclosure.

Exemplary formulations including nanoparticles as described herein include aqueous suspensions, hydrogel-embedded nanoparticles, polymer-based films or coatings, sprayable formulations, micellar or liposomal formulations, lyophilized powders, paste or gel formulations, self-assembling nanoparticle systems, nanoparticle-antibiotic conjugates and the like. It is to be understood that this list is not exhaustive and that other formulations will become apparent to one of skill based on the present disclosure.

Exemplary substrates include implants and implantable materials. Exemplary substrates include tracheal tubes, endotracheal tubes, vascular grafts, stents, pacemaker leads, defibrillator components, catheters (e.g., urinary, central venous, or peritoneal dialysis catheters), orthopedic implants such as bone screws, plates, rods, and joint prostheses, dental implants, craniofacial implants, cochlear implants, spinal cages, intervertebral discs, surgical meshes, hernia repair meshes, breast implants, intraocular lenses, and bioscaffolds for tissue engineering and the like. It is to be understood that this list is not exhaustive and that implants will become apparent to one of skill based on the present disclosure. The substrate may also include absorbable or non-absorbable sutures, hemostatic materials, wound closure devices, or barrier membranes. In some embodiments, the substrate may comprise biodegradable or resorbable materials, such as polylactic acid (PLA), polyglycolic acid (PGA), polycaprolactone (PCL), or poly(lactic-co-glycolic acid) (PLGA), while in other embodiments, the substrate may be metallic (e.g., titanium, stainless steel, nitinol), ceramic, or composite in nature.

Exemplary substrates include tissue, such as tissue where a wound is present of a subject. A layer of the nanoparticles can be provided on the tissue and the wound of the subject in a manner so as to inhibit or prevent the formation of a bacterial biofilm, as described herein.

In accordance with certain examples, nanoparticles as described herein can be incorporated into pharmaceutical compositions suitable for administration. Such pharmaceutical compositions typically comprise the nanoparticles disclosed herein and a pharmaceutically acceptable carrier. As used herein the term “pharmaceutically acceptable carrier” is intended to include any and all solvents, dispersion media, coatings, antibacterial and antifungal agents, isotonic and absorption delaying agents, wound dressings, and the like, compatible with pharmaceutical administration. The use of such media and agents for pharmaceutically active substances is well known in the art. Except insofar as any conventional media or agent is incompatible with the nanoparticles or active agent, use thereof in the compositions is contemplated. Supplementary active compounds can also be incorporated into the compositions.

In accordance with certain examples, a pharmaceutical composition of the invention is formulated to be compatible with its intended route of administration, such as topically to tissue or a wound. For example, solutions or suspensions used for topical administration can include the following components: a sterile diluent such as water, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

As used herein, a subject is intended to include both human and non-human mammals. Examples of non-human mammals include, but are not limited to, non-human primates, horses, cows, goats, sheep, dogs, cats, mice, rats, hamsters, guinea pigs and the like.

### IX. Kits

In accordance with certain other examples, kits for treating bacterial infections in a subject are provided. A “kit” is a collection of parts forming the kit. In one example, the kit may include nanoparticles as described herein in a vial. The kit may include a pharmaceutically acceptable carrier in a vial. The kit may include nanoparticles in a pharmaceutically acceptable carrier in a vial. In an additional example, the kit may also include instructions for inhibiting or preventing bacterial biofilm formation. In some examples, the kit may also comprise, e.g., a buffering agent, a preservative, or a protein stabilizing agent. In some examples, the kit may include an apparatus for administering the nanoparticles such as a spray device or other apparatus for topical administration to a substrate. Other suitable components for including in the kit will be selected by the person of ordinary skill in the art, given the benefit of this disclosure.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Synthesis of Gelatin Nanoparticles With Copolymer Conjugated Thereto Via an Acid Labile Bond

Gelatin nanoparticles (GNPs) were synthesized using Gelatin type B bloom 225 via a two-step dissolution method and crosslinked using glutaraldehyde. See Coester, C. J.; Langer, K.; van Briesen, H.; Kreuter, J. Gelatin nanoparticles by two step desolvation-a new preparation method, surface modifications and cell uptake. Journal of Microencapsulation 2000, 17, 187-193. The two-step desolvation method removes the lower molecular weight gelatin fractions, leaving only the high molecular weight gelatin. The pH was adjusted to 3.0 and the solution was left stirring at room temperature for two days before purification via centrifugation. This two-step process ensures smaller and more uniform particles compared to the single desolvation process to prepare gelatin nanoparticles.

The copolymer, methoxy PEG-benzoic-imine-branched polyethyleneimine (mPEG-b-i-bPEI) was synthesized as previously reported. See Deirram, N.; Zhang, C.; Kermaniyan, S. S.; Johnston, A. P. R.; Such, G. K. pH-Responsive Polymer Nanoparticles for Drug Delivery. Macromolecular Rapid Communications 2019, 40, e1800917. PEG-b-i-PEI co-polymer was conjugated to gelation nanoparticle (GNP) via a two-step coupling reaction using 1-ethyl-3-(3-dimethylamino) propyl carbodiimide hydrochloride (EDC) and N-hydroxysuccinimide (NHS) to create a polymer conjugated gelatin nanoparticle (P-GNP). In this EDC/NHS reaction, carboxyl groups present on gelatin interact with EDC to form a reactive intermediate, which then reacts with NHS to form stable esters on the gelatin. Then, the copolymer is introduced, and the primary amines on the copolymer react with the esters now present on the gelatin NPs to form strong amide bonds between the copolymer and gelatin. The amide bonds are acid labile through hydrolysis under acidic conditions. The amount of copolymer conjugated was quantified via optical density measurement using a standard curve. The nanoparticles are free of an antibiotic.

According to one aspect, antibiotic-free nanoparticles that exhibit antibiofilm and therefore antibacterial properties against S. epidermidis were prepared. TEM images indicate uniform, spherical shapes (FIGS. 2B and 2C). FTIR analysis showed a characteristic peak corresponding to the C—O—C stretching vibration at 1100 cm⁻¹, which appears in the copolymer and P-GNP spectrum but is absent in the GNP spectrum, further confirming the conjugation of the copolymer to the GNPs (FIGS. 2B and 2D). DLS analysis showed a significant increase in the NP size and polydispersity index (PDI), indicating successful copolymer conjugation (FIGS. 2B and 2C). Additionally, both the P-GNP and GNP formulations exhibited a net positive zeta potential when suspended in water (FIG. 2E). The hydrodynamic diameter, polydispersity index, and zeta (ζ)-potential of the nanoparticle formulations were obtained over multiple days to confirm the nanoparticle stability. Samples of the nanoparticle formulations were stored in buffers with varying pHs to investigate the swelling or degradation of the nanoparticles in response to pH. In water, the nanoparticles remained stable over 3 days, with no significant difference observed. In PBS and NaCl solutions, the nanoparticle formulations exhibited the greatest instability, confirming the pH-responsiveness of the nanoparticles to detach polymer from nanoparticle cores. The stability study also ensured the particles remained effective for use within 3 days of synthesis while being stored at 4 degrees Celsius.

### Example II

### Bacterial Strains and Growth Media

S. epidermidis American Type Culture Collection (ATCC) 12228 were routinely cultured in TSB broth (Thermo Scientific, USA, containing 17.0 g/L soytone, 3.0 g/L glucose, 2.5 g/L sodium chloride and 5.0 g/L dipotassium phosphate). Overnight cultures S. epidermidis were prepared by inoculating bacterial glycerol stock into TSB followed by incubation at 37° C. with orbital shaking at 200 rpm for 16-18 h. The overnight culture was used to inoculate fresh nutrient-rich medium at a ratio of 1:500 (v:v) and incubated at 37° C. with shaking at 200 rpm until reaching the exponential phase. The growth phase was monitored by measuring the optical density at 600 nm (OD₆₀₀) using a spectrophotometer until it reached a value of OD₆₀₀−0.1. Bacteria biofilms were prepared by inoculating 1×10⁶ colony forming units per mL of culture (CFU/mL) exponential phase bacteria in TSBG and formed by static incubation at 37° C. for 24 h.

### Example III

### Biofilm Inhibition Assay

The effect of the nanoparticles on bacterial adhesion and biofilm formation was analyzed via CV staining. Briefly, biofilms were formed as previously described with the concurrent addition of treatments. Therapeutics (P-GNP), positive controls (PC) (only bacteria, no treatment), negative controls (NC) (media only), therapeutic controls (TC) (co-polymer alone), and therapeutic negative controls (TNC) (media with therapeutics) were included. After 24 hours incubation at 37° C., the supernatant was removed, and biofilms were washed with 1×PBS. The biomass was washed once with 1×PBS, fixed with 100% methanol and stained with a 0.1% (w/v) CV solution. The CV solution was removed post staining, and each well was washed 3 times with 1×PBS and eluted with 100% methanol. The OD of the CV stain was measured at 590 nm (OD₅₉₀), where a higher absorbance reading corresponds with greater biomass. The percentage of remaining biofilm biomass was quantified using Equation [1]. The lowest P-GNP concentration that inhibits bacteria biofilm growth was taken as the minimal biofilm inhibitory concentration (MBIC).

$$Normalized biofilm biomass ( \% ) = \frac{{OD}_{550 , sample} - {OD}_{550 , drugNC} - {OD}_{550 , NC}}{{OD}_{550 , PC} - {OD}_{550 , NC}} \times 100 \qquad ( 1 )$$

### Example IV

### Minimum Inhibitory Concentration (MIC) Quantification

S. epidermidis were seeded at a final concentration of 1×10⁶ CFU/mL in their exponential growth phase (Optical density at 600 nm (OD₆₀₀)=0.1 (OD₆₀₀=0.1)) and added to a plate containing serially diluted PC, NC, TC, and TNC. Samples were incubated at 37° C. for 16-18 hours with orbital shaking at 200 rpm. OD₆₀₀ were measured, and bacteria growth were normalized using Equation (2).

$$Normalized growth ( \% ) = \frac{{OD}_{600 , sample} - {OD}_{600 , TNC}}{{OD}_{600 , PC} - {OD}_{600 , NC}} \times 100 \qquad ( 2 )$$

### Example V

### Biofilm Confocal Imaging

Biofilms were cultured on silicon wafers, replicating the conditions outlined previously. Following a 24-hour exposure to the therapeutic compounds and controls at 37° C., the treatments were discarded, and the biofilms were rinsed with 1×PBS. Subsequently, biofilms were stained utilizing LIVE/DEAD BacLight Bacterial Viability dyes (Thermo Fisher Scientific, USA) following the manufacturer's protocol. The stained biofilms were visualized using a 63× water immersion lens on the Opera Phenix Plus High-Content Screening System (PerkinElmer, Waltham, MA). For image analysis, Harmony high-content analysis software was employed. Image acquisition was executed at 0.5 μm steps to a maximal depth of 20 μm from the base, across six regions measuring 40,000 μm² within quadruplicate wells.

### Example VI

### P-GNP Anti-Biofilm Adhesion Properties

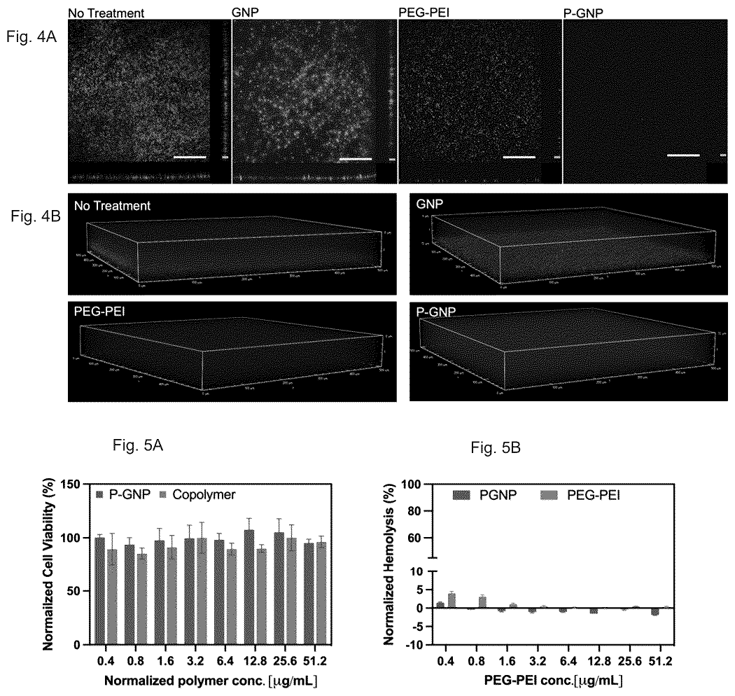

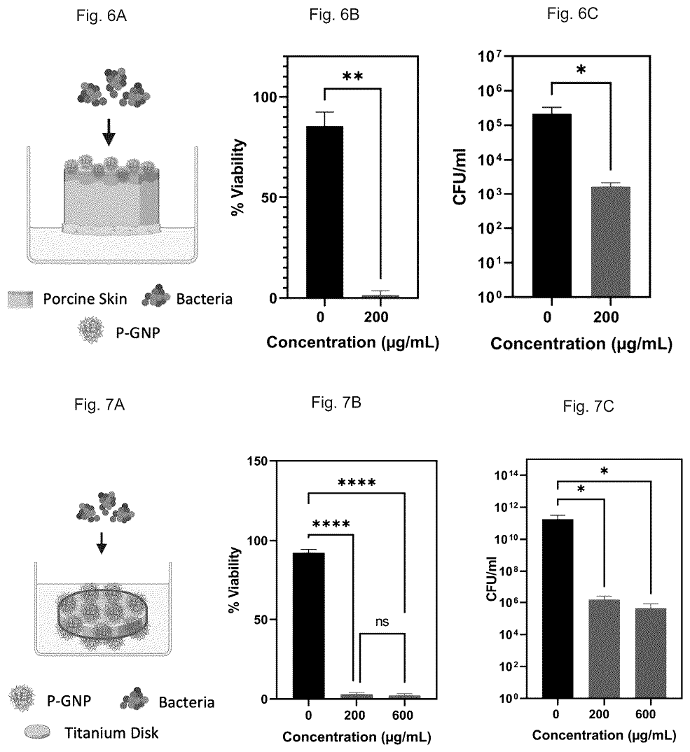

Using LIVE/DEAD viability staining, the viability of the bacteria following treatment was also assessed. With reference to FIG. 4a to FIG. 4b, the SYTO 9 stains all bacterial cells green and the propidium iodide only labels damaged bacterial membranes in red. The PBS control appears completely green because no treatment was given to the biofilm. However, when planktonic bacteria and P-GNP is incubated together overnight together with either P-GNP or the free copolymer, there is a mixture of red and green, indicating a mixture of live and membrane-damaged bacteria. Therefore, P-GNP disrupts the S. epidermidis biofilm to a greater extent as compared to free copolymer.

### Example VII

### Biofilm SEM Imaging

Before SEM imaging, biofilms were formed on silicon wafers under the same conditions as described above. The biofilm samples were treated by being incubated with various NP formulations and control groups and gently washed three times with 1×PBS. The samples were then fixed with a 2% (v/v) glutaraldehyde (GA), 2% (v/v) paraformaldehyde, in 1×PBS solution at 4° C. for 4 h. After washing with 1×PBS, the samples were dehydrated by incubating with ascending ethanol (Greenfield Global, Brookfield, CT) series (50%, 60%, 70%, 80%, 90%, and 100% (v/v)) for 10 min each at room temperature. Samples were air-dried overnight at room temperature and gold-palladium was sputter-coated at 20 mA for 60 s with a sputter coater (Jeol JFC-1600). Samples were then imaged with a SEM (Quattro S, Thermo Fisher Scientific, USA) operated at 2 kV.

### Example VIII

### Cytotoxicity Assay: Hemolysis Assay

Single-donor human red blood cells were used for hemolysis assays (Innovative Research, USA). The potential cytotoxicity of the P-GNPs was tested using the hemolysis test against 5% (v/v) red blood cells in 1×PBS. A positive control (i.e., 100% lysis) with 1% (v/v) Triton X-100 (Sigma-Aldrich, USA) and a negative control (i.e., 0% lysis) with 1×PBS were included. After a 2-hour incubation at 37° C. with 100 rpm orbital shaking, the released hemoglobin was measured at 540 nm (OD₅₄₀), and the % hemolysis was quantified using Equation 3.

$$Normalized viability ( \% ) = \frac{{OD}_{540 , Therapeutic} - {OD}_{540 , NC}}{\overline{{OD}_{540 , PC} - {OD}_{540 , NC}}} \times 100 \qquad ( 3 )$$

### Example IX

### Cytotoxicity Assay: CCK-8 Assay

Murine fibroblast cells (NIH3T3) and human endothelial cells (HUVEC) were used for the cytotoxicity assay (American Type Culture Collection (ATCC), USA). Fibroblasts were seeded in DMEM/F12 supplemented with 10% (v/v) fetal bovine serum (FBS) and 1% penicillin-streptomycin. Cells were seeded at 5000 cells/well in 96-well tissue culture plates. Following 24-hr incubation at 37° C. with 5% CO₂, cells were treated with NP formulations (i.e., GNP and P-GNP) at varies concentration and 10% (v/v) dimethyl sulfoxide (DMSO) as experimental control (i.e., representing cell death). A positive control (PC, cells treated with 1×PBS) as 100% viable, and A negative control (NC, media only without cell) were included. Following a 24-hour incubation, cells were washed three times with 1×PBS and incubated with media containing 10% (v/v) CCK-8 (ApexBio, USA). After a 2-hour incubation at 37° C. with 5% CO₂, absorbance was measured at 450 nm (OD₄₅₀), and viability was calculated using Equation 4.

$$Normalized viability ( \% ) = \frac{{OD}_{450 , Therapeutic} - {OD}_{450 , NC}}{\overline{{OD}_{450 , PC} - {OD}_{450 , NC}}} \times 100 \qquad ( 4 )$$

### Example X

### In Vitro Cell Viability and Cytotoxicity

In addition to the antimicrobial and antibiofilm studies, the cytotoxicity of P-GNP and copolymer were examined against mammalian cells (i.e., fibroblast, NIH 3T3) and red blood cells. The cell metabolic was monitored via cell counting kit-8 (CCK-8), and results indicated that all tested formulations demonstrated minimal cytotoxicity to fibroblast cells (FIG. 5A).

The hemolytic activity of the NP formulation was investigated using a hemolysis assay by incubating human red blood cells with different concentrations of treatments for 2 hours. Minimal hemolysis was observed for NP formulations and the free co-polymer. P-GNP, at all tested concentrations, exhibited excellent hemocompatibility with human red blood cells and is not toxic to mammalian cells.

### Example XI

### Ex Vivo Studies

Frozen porcine skin tissue was thawed and washed with 1×PBS. A biopsy punch (8 mm) was used to create tissue sections from freshly thawed tissue. The tissue pieces were sterilized by immersing in 70% ethanol for 20 minutes, put under UV light, and dried for 30 minutes in a CellGard™M Energy Saver class II, type A2 biosafety cabinet (NuAire, Plymouth, MN). The sterile tissue was then placed on Baird-Parker agar plates containing 1.5% (w/v) agar. The skin was first treated with an aqueous formulation of P-GNP at concentrations of 100 and 200 μg/mL and was allowed to dry for 3 hours to form a layer. Then, the skin was infected by adding 10 μL of S. epidermidis suspension (OD₆₀₀=0.1) on top of the 8 mm skin sample, with the P-GNP layer between the S. epidermidis and the skin. The inoculated skin pieces were incubated for 24 h at 37° C. in a humidified chamber. Controls of tissue inoculated with bacteria only and tissue without bacteria and without treatment were included. After incubation, skin samples were washed with 1×PBS and homogenized using a gentle MACS™ dissociator (Miltenyi Biotec, Waltham, MA). The homogenized samples were serially diluted 10-fold and each dilution was plated onto agar plates. The plates were incubated at 37° C. for 12 hour followed by CFU enumeration and an XTT ((2,3-Bis-(2-Methoxy-4-nitro-5-sulfophenyl)-2H-tetrazolium-5-carboxanilide)) assay to determine bacterial cell viability. SEM images of the skin samples were then taken after glutaraldehyde fixation, ethanol dilutions, critical point drying, and sputter coating.

According to one aspect, porcine skin was first sterilized before the addition of P-GNP treatment, which serves as a preventative treatment to inhibit bacteria adhesion and biofilm formation. After a three-hour incubation with the treatment, S. epidermidis was inoculated onto the skin. Various concentrations of P-GNP were evaluated. Compared to skin samples with only bacteria and without P-GNP treatment, a 1000-fold reduction in CFU resulted after treatment with the P-GNP formulation at concentration 200 micrograms/ml. To quantify the viability of bacterial cells from the skin samples, an XTT assay was performed using the homogenized skin sample solution. The XTT results indicate approximately 3% bacterial viability with P-GNP treatment, i.e., 3% adhesion with remaining bacteria being washed away, compared to approximately 90% bacterial cell viability in the inoculated sample without P-GNP treatment. SEM imaging was performed to better analyze the effect of P-GNP in this ex vivo model and indicated significant reduction in S. epidermidis bacteria found within the tissue environment.

### Example XII

### Antimicrobial Properties of Gelatin Nanoparticles With Copolymer Conjugated Thereto

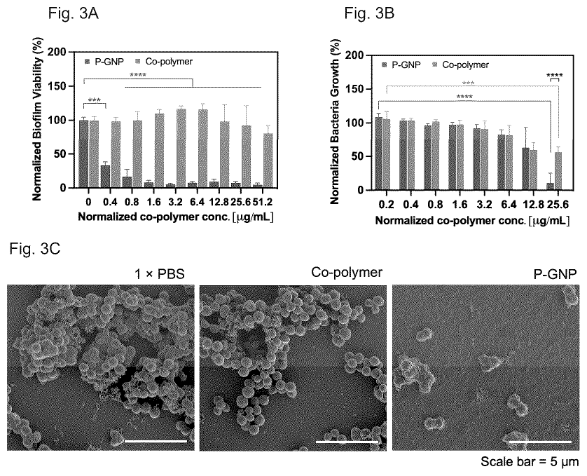

Gelatin nanoparticles with copolymer conjugated thereto (P-GNP) prepared as described herein were tested for antimicrobial properties as described herein. The P-GNPs exhibited significant antibacterial activity against S. epidermidis in a dose-dependent manner. P-GNPs demonstrated exceptional antibiofilm activity, significantly reducing S. epidermidis biofilm viability to below 50% at concentrations as low as 0.4 g/mL (FIG. 3A). At higher concentrations (25.6 μg/mL), biofilm viability dropped to less than 20%, indicating a reduction in bacterial adhesion and a potent disruption of biofilm structure. In contrast, copolymer alone had minimal effects on biofilm viability, even at the highest tested concentrations, demonstrating efficacy of the P-GNP system. The minimal effects observed with the copolymer alone confirm that the polymer coating does not possess inherent antibacterial or antibiofilm properties, emphasizing the importance of the combination of the gelatin nanoparticle core and its polymer coating.

Furthermore, bacterial growth was reduced approximately 60% at the highest concentration of 25.6 μg/mL, whereas the copolymer alone showed less antibacterial effects across all tested concentrations compared to the P-GNP treated groups (FIG. 3B). Accordingly, aspects of the present disclosure are directed to a gelatin nanoparticle having a PEG copolymer conjugated thereto and which demonstrates antibiofilm and antibacterial efficacy. The gelatin nanoparticle having a PEG copolymer conjugated thereto exhibits superior antibacterial and antibiofilm efficacy compared to the copolymer or uncoated GNPs.

SEM images further confirmed the disruptive effects of P-GNPs on S. epidermidis biofilms. Untreated biofilms (1×PBS) showed dense clusters of bacterial cells embedded within a robust extracellular matrix. Similarly, treatments with copolymer or uncoated GNPs resulted in little to no visible disruption, with intact bacterial aggregates persisting. In contrast, P-GNP treatment led to significant degradation of the biofilm matrix (FIG. 3C). Accordingly, the present disclosure provides P-GNPs for use in therapeutic methods for treating biofilm-associated infections.

### Example XIII

### Metal Implant Model

S. epidermidis is one of the most common causes of infections on implanted devices. Accordingly, P-GNPs as described herein were tested on an implant model. Metal squares approximately 5 mL in length were first plasma-coated to induce a negative surface charge, to enhance the electrostatic interaction with the nanoparticles. Then, the metal squares were incubated in a 96-well plate with 200 microliters of P-GNP for 3 hours. The metal samples were then allowed to dry for 1 hour forming a layer of P-GNP. S. epidermidis was then inoculated onto the surface of the metal sample, with the layer of P-GNP between the bacteria and the substrate surface. After 3 hours, the surface was washed with 1×PBS to mimic biofilm inhibitory conditions. The squares were then allowed to dry before incubating overnight on Baird-Parker agar plates. After 12-16 hours, the metal squares were placed in 1 mL of PBS and homogenized. The solution was then tested with XTT assays and CFU counting by plating dilutions of the homogenized solution onto Baird-Parker agar plates. After 12-16 hours of incubation, the CFUs were counted. Bacterial burden on titanium disks was measured by colony-forming units (CFU/mL). Both 200 and 600 μg/mL P-GNP treatments led to a significant reduction in viable bacteria compared to control (*p<0.05), with CFU levels reduced by several orders of magnitude, supporting potent antibiofilm activity. According to one aspect, the present disclosure contemplates the use of P-GNP in a clinical setting as an implant coating to prevent the formation of S. epidermidis biofilms.

### Example XIV

### Antimicrobial Properties of Gelatin Nanoparticles With Copolymer Conjugated Thereto Against P. aeruginosa

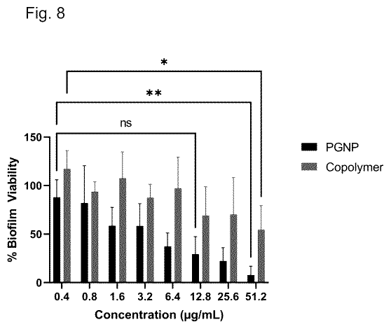

P. auerginosa biofilm inhibitory efficacy was assessed following 24-hour incubation with increasing concentrations (0.4-51.2 μg/mL) of P-GNP or the free copolymer (FIG. 8). Results are expressed as a percentage of biofilm viability relative to untreated control. P-GNP treatment resulted in a significant, concentration-dependent reduction in P. aeruginosa biofilm viability, with notable reductions observed at concentrations≥3.2 μg/mL. In contrast, the free copolymer demonstrated only modest antibiofilm activity across all concentrations tested. At concentrations of 6.4 μg/mL and above, P-GNP significantly outperformed the copolymer in reducing biofilm viability, indicating enhanced efficacy of the nanoparticle formulation. The minimal biofilm inhibitory concentration (MBIC) for P-GNP appears to lie between 3.2-6.4 μg/mL.

### Example XV

### Embodiments

Embodiments of the present disclosure are directed to a method of preventing or inhibiting bacterial biofilm formation on a substrate including the steps of providing the substrate with a layer of nanoparticles, wherein the nanoparticles comprise a core comprising gelatin, and a layer of copolymer attached to the core by an acid labile bond, wherein bacteria contacts the layer on the substrate and the bacteria creates an environment comprising an acidic pH and a gelatinase, wherein the acid labile bond is broken thereby releasing the copolymer from the core causing degradation of the layer, wherein the gelatinase degrades the core into portions of the core causing degradation of the layer, and wherein degradation of the layer inhibits or prevents bacterial adhesion to the substrate and/or bacterial biofilm formation on the substrate, and wherein proliferation of the bacteria is inhibited or prevented. According to one aspect, the copolymer is a PEG-PEI copolymer linked or conjugated to the core via the acid labile bond. According to one aspect, the copolymer is a poly(ethylene glycol) (PEG) derivative linked or conjugated to the core via the acid labile bond. According to one aspect, the copolymer is methoxy PEG-benzoic-imine-branched polyethyleneimine (mPEG-b-i-bPEI) linked ot conjugated to the core via the acid labile bond. According to one aspect, a nanoparticle is provided that includes a core comprising gelatin optionally lacking an antimicrobial agent, and a pH-degradable poly(ethylene glycol) (PEG) derivative copolymer attached to the core via an acid labile bond. According to one aspect, the pH-degradable poly(ethylene glycol) (PEG) derivative copolymer comprises a copolymer of PEG and PEI. According to one aspect, the pH-degradable material is a methoxy PEG-benzoic-imine-branched polyethyleneimine (mPEG-b-i-bPEI). According to one aspect, the nanoparticle further includes a nuclease. According to one aspect, the nanoparticle lacks or excludes an antimicrobial agent toxic to bacteria. According to one aspect, a pharmaceutical composition is provided that includes a nanoparticle that includes a core comprising gelatin optionally lacking an antimicrobial agent, and a pH-degradable poly(ethylene glycol) (PEG) derivative copolymer attached to the core via an acid labile bond, and a pharmaceutically acceptable excipient. According to one aspect, a method of inhibiting or preventing a bacterial infection in a subject is provided that includes implanting an implant within the subject, wherein the implant has a coating of a nanoparticle that includes a core comprising gelatin optionally lacking an antimicrobial agent, and a pH-degradable poly(ethylene glycol) (PEG) derivative copolymer attached to the core via an acid labile bond, wherein bacterial infection on the implant is prevented, reduced, inhibited or eradicated. According to one aspect, a method of inhibiting or preventing a bacterial infection in wound of a subject is provided including topically administering a nanoparticle that includes a core comprising gelatin optionally lacking an antimicrobial agent, and a pH-degradable poly(ethylene glycol) (PEG) derivative copolymer attached to the core via an acid labile bond, to the wound of the subject, wherein bacterial infection on the wound is prevented, reduced, inhibited or eradicated. According to one aspect, the subject is a mammal. According to one aspect, the subject is a human. According to one aspect, a method of making a nanoparticle is provided that includes the steps of providing a gelatin core excluding an antibiotic therein, and attaching a methoxy PEG-benzoic-imine-branched polyethyleneimine (mPEG-b-i-bPEI) to the gelatin core by an acid labile bond.

1. A method of preventing or inhibiting bacterial biofilm formation on a substrate comprising
providing the substrate with a layer of nanoparticles, wherein the nanoparticles comprise a core comprising gelatin, and a layer of copolymer attached to the core by an acid labile bond,
wherein bacteria contacts the layer on the substrate and the bacteria creates an environment comprising an acidic pH and a gelatinase,
wherein the acid labile bond is broken thereby releasing the copolymer from the core causing degradation of the layer,
wherein the gelatinase degrades the core into portions of the core causing degradation of the layer, and
wherein degradation of the layer inhibits or prevents bacterial adhesion to the substrate and/or bacterial biofilm formation on the substrate, and
wherein proliferation of the bacteria is inhibited or prevented.

2. The method of claim 1 wherein the copolymer is a PEG-PEI copolymer linked or conjugated to the core via the acid labile bond.

3. The method of claim 1 wherein the copolymer is a poly(ethylene glycol) (PEG) derivative linked or conjugated to the core via the acid labile bond.

4. The method of claim 1 wherein the copolymer is methoxy PEG-benzoic-imine-branched polyethyleneimine (mPEG-b-i-bPEI) linked ot conjugated to the core via the acid labile bond.

5. A nanoparticle comprising
a core comprising gelatin lacking an antimicrobial agent, and
a pH-degradable poly(ethylene glycol) (PEG) derivative copolymer attached to the core via an acid labile bond.

6. The nanoparticle of claim 5 wherein the pH-degradable poly(ethylene glycol) (PEG) derivative copolymer comprises a copolymer of PEG and PEI.

7. The nanoparticle of claim 5 wherein the pH-degradable material is a methoxy PEG-benzoic-imine-branched polyethyleneimine (mPEG-b-i-bPEI).

8. The nanoparticle of claim 5 further comprising a nuclease.

9. A pharmaceutical composition comprising
a nanoparticle of claim 5 and a pharmaceutically acceptable excipient.

10. A method of inhibiting or preventing a bacterial infection in a subject comprising
implanting an implant within the subject, wherein the implant has a coating of a nanoparticle of claim 5, wherein bacterial infection on the implant is prevented, reduced, inhibited or eradicated.

11. A method of inhibiting or preventing a bacterial infection in wound of a subject comprising
topically administering a nanoparticle of claim 5 to the wound of the subject, wherein bacterial infection on the wound is prevented, reduced, inhibited or eradicated.

12. The method of claim 10 wherein the subject is a mammal.

13. The method of claim 10 wherein the subject is a human.

14. A method of making a nanoparticle comprising
providing a gelatin core excluding an antibiotic therein, and
attaching a methoxy PEG-benzoic-imine-branched polyethyleneimine (mPEG-b-i-bPEI) to the gelatin core by an acid labile bond.
